(19) United States
(12) Patent Application Publication
(10) Pub. No.: US 2022/0296407 A1
(43) Pub. Date: Sep. 22, 2022

(54) Ostomy Bag Assembly

(71) Applicant: Justin Sims, San Francisco, CA (US)

(72) Inventor: Justin Sims, San Francisco, CA (US)

(21) Appl. No.: 17/204,616

(22) Filed: Mar. 17, 2021

(51) Int. Cl. A61F 5/441; A61F 5/44

(52) U.S. Cl. CPC A61F 5/441; A61F 5/4404; A61F 5/4405

(57) ABSTRACT

An ostomy bag assembly for facilitating an ostomy bag to be drained without disconnecting the ostomy bag from a catheter includes an ostomy bag that has an inlet tube and a drain valve. An air freshening layer is provided that surrounds the ostomy bag. The air freshening layer is infused with a chemical deodorant to conceal odors associated with ostomy bags. A cover is positioned around the ostomy bag and the cover is comprised of a fluid impermeable material to contain leaks from the ostomy bag. A support is provided that removably engages the sleeve for hanging the sleeve from a structure.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to ostomy devices and more particularly pertains to a new ostomy device for facilitating an ostomy bag to be drained without disconnecting the ostomy bag from a catheter.

### (2) Description of Related Art Including Information Disclosed Under 37 CFR 1.97 and 1.98

The prior art relates to ostomy devices including a colostomy bag that has a gas valve for releasing gases trapped in a colostomy bag. The prior art discloses a deodorant that is positioned in an ostomy bag for concealing odors associated with ostomy bags. The prior art discloses an ostomy bag that has a zip-lock type closure for draining the ostomy bag. The prior art discloses a colostomy bag that has an irrigation input to facilitate an interior of the colostomy bag to be irrigated while the colostomy bag remains in place on a user. The prior art discloses an ostomy bag drainage device that includes a vacuum source and a reservoir for suctionally draining an ostomy bag. The prior art discloses a variety of ostomy bags that have drain portions structurally integrated therein for draining the ostomy bags.

### BRIEF SUMMARY OF THE INVENTION

An embodiment of the disclosure meets the needs presented above by generally comprising an ostomy bag that has an inlet tube and a drain valve. An air freshening layer is provided that surrounds the ostomy bag. The air freshening layer is infused with a chemical deodorant to conceal odors associated with ostomy bags. A cover is positioned around the ostomy bag and the cover is comprised of a fluid impermeable material to contain leaks from the ostomy bag. A support is provided that removably engages the sleeve for hanging the sleeve from a structure.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of an ostomy bag assembly according to an embodiment of the disclosure.

FIG. 2 is a back view of an embodiment of the disclosure.

FIG. 3 is a front cut-away view of an embodiment of the disclosure.

FIG. 4 is a cross sectional view taken along line 4-4 of FIG. 2 of an embodiment of the disclosure.

FIG. 5 is a front view of an alternative embodiment of the disclosure.

### DETAILED DESCRIPTION OF THE INVENTION

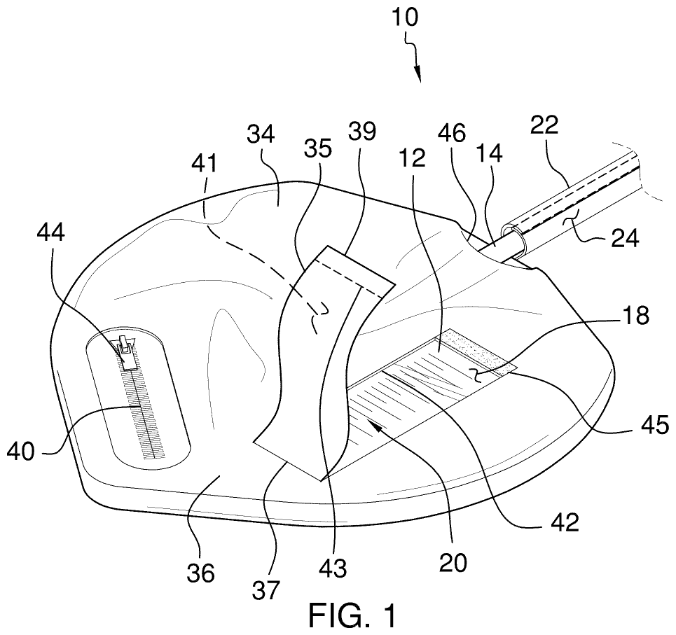

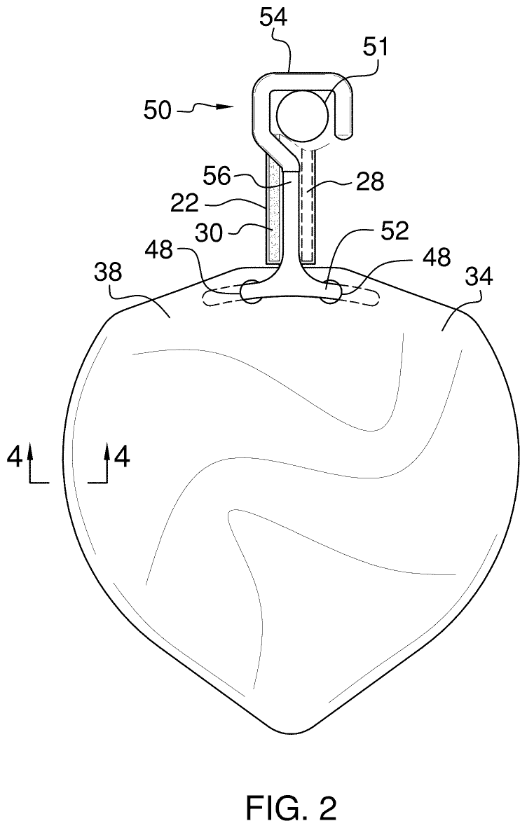

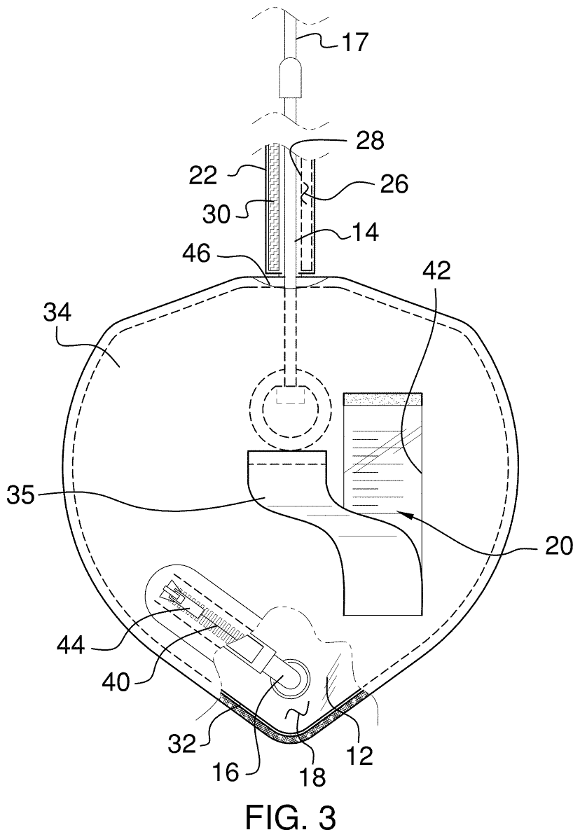

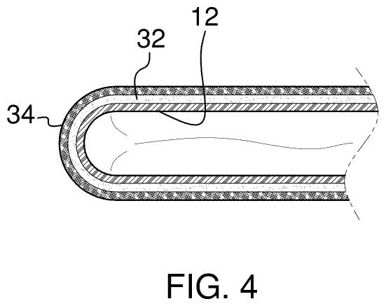

With reference now to the drawings, and in particular to FIGS. 1 through 5 thereof, a new ostomy device embodying the principles and concepts of an embodiment of the disclosure and generally designated by the reference numeral 10 will be described.

As best illustrated in FIGS. 1 through 5, the ostomy bag assembly 10 generally comprises an ostomy bag 12 that has an inlet tube 14 and a drain valve 16. The inlet tube 14 is fluidly attachable to a catheter 17 to contain human waste. The drain valve 16 is actuatable between an open condition for draining the ostomy bag 12 and a closed condition for retaining contents in the ostomy bag 12. The ostomy bag 12 is comprised of a translucent material to facilitate contents of the ostomy bag 12 to be visible. The ostomy bag 12 has an outer surface 18 and the outer surface 18 has volume indicia 20 printed thereon. The volume indicia 20 comprise a graduated scale for displaying the volume of the contents of the ostomy bag 12.

A sleeve 22 is provided and the sleeve 22 is wrappable around the inlet tube 14. The sleeve 22 is comprised of an opaque material to inhibit the inlet tube 14 from being visible, and the sleeve 22 has a first surface 24 and a second surface 26. A first mating member 28 is coupled to the first surface 24 of the sleeve 22 and a second mating member 30 is coupled to the second surface of the sleeve 22. The first mating member 28 is matable to the second mating member 30 for forming the sleeve 22 into a closed loop to retain the sleeve 22 around the inlet tube 14. Each of the first mating member 28 and the second mating member 30 may comprise complementary portions of a hook and loop fastener.

An air freshening layer 32 is provided and the air freshener surrounds the ostomy bag 12. The air freshening layer 32 is infused with a chemical deodorant to conceal odors associated with ostomy bags. The air freshening layer 32 rests against the outer surface 18 of the ostomy bag 12 and the air freshening layer 32 substantially covers the outer surface 18. The volume indicia 20 are exposed by the air freshening layer 32 thereby facilitating the volume indicia 20 to be visible. The chemical deodorant may comprise an institutional deodorant or the like.

A cover 34 is provided and the cover 34 is positioned around the ostomy bag 12. The cover 34 is comprised of a fluid impermeable material to contain leaks from the ostomy bag 12. The cover 34 completely surrounds the ostomy bag 12 having the air freshening layer 32 being positioned between the ostomy bag 12 and the cover 34. The cover 34 has a front side 36 and a back side 38, and the front side 36 has a first opening 40 extending therethrough. The first opening 40 is aligned with the drain valve 16 thereby facilitating the drain valve 16 to be accessible through the cover 34.

The front side 36 has a second opening 42 extending therethrough and the second opening 42 is aligned with the volume indicia 20 on the ostomy bag 12 thereby facilitating the volume indicia 20 to be visible. A closure 44 is coupled to the ostomy bag 12 and the closure 44 is aligned with the first opening 40. The closure 44 is positionable in an open position for accessing the drain valve 16 and a closed position for covering the drain valve 16. The closure 44 may comprise a zipper or other similar type of elongated closure.

A flap 35 is provided that has a first end 37, a second end 39 and a first surface 41 extending therebetween. The first end 37 is coupled to the front side 36 of the cover 34 having the cover 34 being aligned with the second opening 42. A first mating member 43 is coupled to the first surface 41 of the flap 35 and the first mating member 43 is aligned with the second end 39. A second mating member 45 is coupled to the front side 36 and the second mating member 45 is aligned with the second opening 42. The flap 35 is positionable to cover the second opening 42 having the first mating member 43 engaging the second mating member 45. In this way the flap 35 inhibits the ostomy bag 12 from being visible through the second opening 42. Each of the first mating member 43 and the second mating member 45 may comprise a hook and loop fastener or other similar type of mating member.

The cover 34 has a third opening 46 extending therethrough and the inlet tube 14 extends through the third opening 46. The third opening 46 is aligned with an intersection between the front side 36 and the back side 38. The back side 38 has a pair of holes 48 each extending therethrough. The holes 48 are spaced apart from each other and are positioned adjacent to the third opening 46.

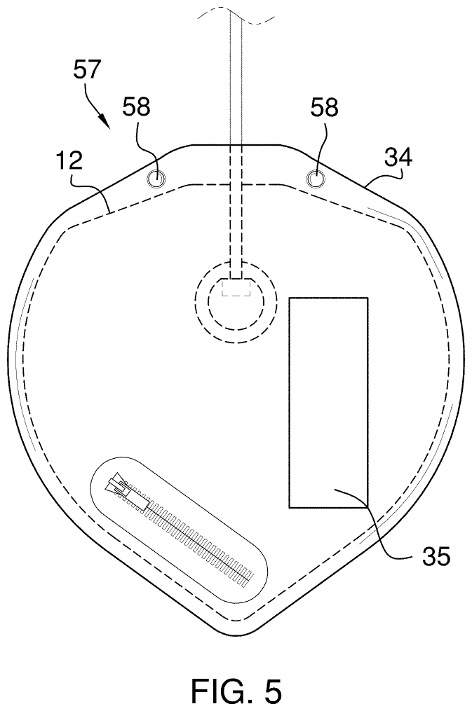

A support 50 is provided that removably engages the cover 34 for hanging the cover 34 from a structure 51. The support 50 has a tail 52, a hook 54 and a central member 56 extending therebetween, and the tail 52 is oriented transverse to the central member 56. The central member 56 extends through the third opening 46 having the tail 52 extending through each of the holes 48. In this way the cover 34 can be attached to the support 50. The hook 54 curves downwardly toward the tail 52 thereby facilitating the hook 54 to engage the structure 51. In an alternative embodiment 57 as is most clearly shown in FIG. 5, the ostomy bag 12 may be removable from the cover 34. The cover 34 may have a pair of fasteners 58 each being coupled thereto for closing the cover 34 around the ostomy bag 12.

In use, the inlet tube 14 is fluidly coupled to the catheter 17 and the support 50 is hung from the structure 51, such as a frame of a wheelchair, a bed frame, or other device employed by a person who uses the ostomy bag 12. The closure 44 is manipulated to access the drain valve 16 and drain the ostomy bag 12. In this way the ostomy bag 12 can be emptied without disconnecting the inlet tube 14 from the catheter. Additionally, the air freshening layer 32 conceals odors commonly associated with the use of an ostomy bag.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An ostomy bag assembly for concealing storage of human waste and concealing odors, said assembly comprising:
an ostomy bag having an inlet tube and a drain valve, said inlet tube is fluidly attachable to a catheter wherein said ostomy bag is configured to contain human waste, said drain valve is actuatable between and open condition for draining said ostomy bag and a closed condition for retaining contents in said ostomy bag;
an air freshening layer surrounding said ostomy bag, said air freshening layer is infused with a chemical deodorant wherein said air freshening layer is configured to conceal odors associated with ostomy bags;
a cover is positioned around said ostomy bag, said cover is comprised of a fluid impermeable material wherein said cover is configured to contain leaks from said ostomy bag, said cover completely surrounding said ostomy bag having said air freshening layer is positioned between said ostomy bag and said cover; and
a support removably engaging said cover for hanging said cover from a structure.

2. The assembly according to claim 1, wherein said ostomy bag is comprised of a translucent material wherein said ostomy bag is configured to facilitate contents of said ostomy bag to be visible, said ostomy bag having an outer surface, said outer surface having volume indicia is printed thereon, said volume indicia comprising a graduated scale for displaying the volume of the contents of said ostomy bag.

3. The assembly according to claim 1, further comprising a sleeve is wrappable around said inlet tube, said sleeve is comprised of an opaque material wherein said sleeve is configured to inhibit said inlet tube from is visible, said sleeve having a first surface and a second surface.

4. The assembly according to claim 3, further comprising:
a first mating member is coupled to said first surface of said sleeve; and
a second mating member is coupled to said second surface of said sleeve, said first mating member is matable to said second mating member for forming said sleeve into a closed loop to retain said sleeve around said inlet tube.

5. The assembly according to claim 2, wherein said air freshening layer rests against said outer surface of said ostomy bag, said air freshening layer substantially covering said outer surface, said volume indicia is exposed by said air freshening layer thereby facilitating said volume indicia to be visible.

6. The assembly according to claim 2, wherein:
said cover has a front side and a back side; and
said front side has a first opening extending therethrough, said first opening is aligned with said drain valve thereby facilitating said drain valve to be accessible through said sleeve.

7. The assembly according to claim 6, wherein said front side has a second opening extending therethrough, said second opening is aligned with said volume indicia on said ostomy bag thereby facilitating said volume indicia to be visible.

8. The assembly according to claim 7, wherein said sleeve has a third opening extending therethrough, said third opening having said inlet tube extending therethrough, said third opening is aligned with an intersection between said front side and said back side.

9. The assembly according to claim 8, wherein said back side has a pair of holes each extending therethrough, said holes is spaced apart from each other and is positioned adjacent to said third opening.

10. The assembly according to claim 9, wherein said support has a tail, a hook and a central member extending therebetween, said tail is oriented transverse to said central member, said central member extending through said third opening having said tail extending through each of said holes thereby facilitating said cover to be attached to said support, said hook curving downwardly toward said tail thereby facilitating said hook to engage the structure.

11. An ostomy bag assembly for concealing storage of human waste and concealing odors, said assembly comprising:
an ostomy bag having an inlet tube and a drain valve, said inlet tube is fluidly attachable to a catheter wherein said ostomy bag is configured to contain human waste, said drain valve is actuatable between and open condition for draining said ostomy bag and a closed condition for retaining contents in said ostomy bag, said ostomy bag is comprised of a translucent material wherein said ostomy bag is configured to facilitate contents of said ostomy bag to be visible, said ostomy bag having an outer surface, said outer surface having volume indicia is printed thereon, said volume indicia comprising a graduated scale for displaying the volume of the contents of said ostomy bag;
a sleeve is wrappable around said inlet tube, said sleeve is comprised of an opaque material wherein said sleeve is configured to inhibit said inlet tube from is visible, said sleeve having a first surface and a second surface;
a first mating member is coupled to said first surface of said sleeve;
a second mating member is coupled to said second surface of said sleeve, said first mating member is matable to said second mating member for forming said sleeve into a closed loop to retain said sleeve around said inlet tube;
an air freshening layer surrounding said ostomy bag, said air freshening layer is infused with a chemical deodorant wherein said air freshening layer is configured to conceal odors associated with ostomy bags, said air freshening layer resting against said outer surface of said ostomy bag, said air freshening layer substantially covering said outer surface, said volume indicia is exposed by said air freshening layer thereby facilitating said volume indicia to be visible;
a cover is positioned around said ostomy bag, said cover is comprised of a fluid impermeable material wherein said cover is configured to contain leaks from said ostomy bag, said cover completely surrounding said ostomy bag having said air freshening layer is positioned between said ostomy bag and said cover, said cover having a front side and a back side, said front side having a first opening extending therethrough, said first opening is aligned with said drain valve thereby facilitating said drain valve to be accessible through said cover, said front side having a second opening extending therethrough, said second opening is aligned with said volume indicia on said ostomy bag thereby facilitating said volume indicia to be visible, said cover having a third opening extending therethrough, said third opening having said inlet tube extending therethrough, said third opening is aligned with an intersection between said front side and said back side, said back side having a pair of holes each extending therethrough, said holes is spaced apart from each other and is positioned adjacent to said third opening; and
a support removably engaging said cover for hanging said cover from a structure, said support having a tail, a hook and a central member extending therebetween, said tail is oriented transverse to said central member, said central member extending through said third opening having said tail extending through each of said holes thereby facilitating said cover to be attached to said support, said hook curving downwardly toward said tail thereby facilitating said hook to engage the structure.
